(19) United States
(12) Patent Application Publication
(10) Pub. No.: US 2026/0013710 A1
(43) Pub. Date: Jan. 15, 2026

(54) MEDICAL SYSTEMS, DEVICES, AND METHODS FOR DUAL LIGHT IMAGING

(71) Applicant: Boston Scientific Scimed, Inc., Maple Grove, MN (US)

(72) Inventors: Kirsten VIERING, Newton, MA (US); Louis J. BARBATO, Franklin, MA (US)

(73) Assignee: Boston Scientific Scimed, Inc., Maple Grove, MN (US)

(21) Appl. No.: 19/260,778

(22) Filed: Jul. 7, 2025

Related U.S. Application Data
(60) Provisional application No. 63/670,263, filed on Jul. 12, 2024.

(51) Int. Cl. A61B 1/04; A61B 1/00

(52) U.S. Cl. CPC A61B 1/042; A61B 1/00009; A61B 1/00045; A61B 1/00087

(57) ABSTRACT

A dual light imaging system includes a medical device having an imaging assembly, a first and second light source, and a computing system. The computing system is configured to perform operations, including: receiving a first image frame captured by the imaging assembly as white light is emitted by the first light source; receiving a second image frame captured by the imaging assembly as narrow band light is emitted by the second light source; dividing the first and second image frame into a plurality of first and second tiles, respectively; determining a first and second intensity profile across the plurality of first and second tiles, respectively; comparing shifts in the second intensity profile to identify a subset of the second tiles corresponding to a region of interest; and using shifts in the first intensity profile as a control to confirm the identified subset correspond to the region of interest.

### CROSS-REFERENCE TO RELATED APPLICATIONS

This application claims the benefit of priority to U.S. Provisional Application No. 63/670,263, filed on Jul. 12, 2024, which is incorporated by reference herein in its entirety.

### TECHNICAL FIELD

The disclosure relates generally to systems, devices, and methods for dual light imaging. More specifically, aspects of the disclosure pertain to systems, devices, and methods for obtaining and processing white light and fluorescence images to facilitate identification of regions of interest.

### BACKGROUND

A medical imaging system may include an imaging device and a white light source integrated with a medical device, such as an endoscope. The endoscope may be inserted into and navigated through a body lumen of a patient to a target site during a diagnostic and/or interventional medical procedure. The white light source may be configured to emit white light onto the target site to illuminate objects and/or features within the target site to facilitate a visualization thereof in images captured by the imaging device. For example, white light imaging performed using the endoscope may allow a physician to visually identify and examine potential regions of interest, including regions of potentially cancerous tissue. Depending on the type and/or extent of cancerous tissue found, endoscopic-guided procedures may be used to resect or remove the affected regions. However, identifying a full extent of the potentially cancerous tissue and/or identifying margins to confirm that the area of tissue immediately surrounding the resected regions is healthy tissue (e.g., non-cancerous) can be challenging using only white light imaging.

### SUMMARY

According to some aspects, the techniques described herein relate to a system for dual light imaging, the system including: a medical device including an imaging assembly configured to capture image frames of a target area; a first light source configured to emit white light; a second light source configured to emit narrow band light; and a computing system communicatively coupled to the imaging assembly, the first light source, and the second light source, and configured to perform operations including: receiving a first image frame captured by the imaging assembly as the computing system controls the first light source to emit the white light onto the target area; receiving a second image frame captured by the imaging assembly as the computing system controls the second light source to emit the narrow band light onto the target area; dividing the first image frame into a plurality of first tiles and the second image frame into a plurality of second tiles; determining a first intensity profile across the plurality of first tiles and a second intensity profile across the plurality of second tiles; comparing shifts in the second intensity profile to identify a subset of the plurality of second tiles corresponding to a region of interest within the target area; and using shifts in the first intensity profile as a control to confirm the subset of the plurality of second tiles correspond to the region of interest.

In some examples, determining the first intensity profile and the second intensity profile may include, for each tile of the plurality of first tiles and the plurality of second tiles, determining a sum of amplitudes for first, second, and third pixels within the respective tile. Comparing the shifts in the second intensity profile may include identifying differences above a predefined threshold in the sum of amplitudes across the plurality of second tiles. Using the shifts in the first intensity profile as a control may include identifying and subtracting differences in the sum of amplitudes across the plurality of first tiles from the differences in the sum of amplitudes across the plurality of second tiles.

In some examples, the operations may further include processing the first image frame to generate a white light image, and processing the second image frame to generate a fluorescence image. The operations may further include providing the white light image and the fluorescence image to a display device communicatively coupled to the computing system for simultaneous display. In other examples, the operations may further include generating a combined image by blending the white light image and the fluorescence image, and providing the combined image to a display device communicatively coupled to the computing system for display. The fluorescence image may depict fluorescence in a first color, and the operations may further include performing a color mapping to change the first color to a second color to increase visibility of the fluorescence in the combined image. In further examples, the operations may further include: identifying a boundary of the region of interest based on the comparing of the shifts in the second intensity profile; smoothing the boundary; and overlaying the boundary on the white light image to generate an annotated white light image.

In some examples, the imaging assembly may include a narrow band filter configured to block a range of wavelengths corresponding to the narrow band light emitted by the second light source from being detected by the imaging assembly. The second light source may be a UV light source configured to emit the narrow band light to excite any fluorescent molecules within the target area, and a re-emission spectrum of the narrow band light upon absorption and re-emission by the fluorescent molecules is shifted and detectable by the imaging assembly.

In some examples, the imaging assembly may include a global shutter configured to control a period of image frame exposure, and the operations may further include switching between controlling the first light source and the second light source at an end of the period of image frame exposure. In other examples, the imaging assembly may include a rolling shutter configured to control a period of image frame exposure, and receiving the second image frame includes: receiving a plurality of second image frames, where, based on the rolling shutter, a portion of the plurality of second image frames include mixed image frames exposed to the white light and the narrow band light as the computing system switches between controlling the first light source and the second light source; and identifying and discarding the mixed image frames.

In some examples, the first light source may be a component of the medical device or the computing system, and the second light source may be a component of the medical device or the computing system. In other examples, the system may be operable in a white light imaging mode, a fluorescence imaging mode, and a hybrid imaging mode.

According to other aspects, the techniques described herein relate to a computing system communicatively coupled to a medical device, including: at least one memory storing instructions; and at least one processor coupled to the at least one memory for executing the instructions to perform operations, the operations including: receiving a first image frame of a target area captured by an imaging assembly of the medical device as a first light source is emitting white light onto the target area; receiving a second image frame of the target area captured by the imaging assembly as a second light source is emitting narrow band light onto the target area; dividing the first image frame into a plurality of first tiles and the second image frame into a plurality of second tiles; for each tile of the plurality of first tiles and the plurality of second tiles, determining a sum of amplitudes for first, second, and third pixels within the respective tile; comparing shifts in the sum of amplitudes across the plurality of second tiles to identify a subset of the plurality of second tiles corresponding to a region of interest within the target area; and using shifts in the sum of amplitudes across the plurality of first tiles as a control to confirm the subset of the plurality of second tiles correspond to the region of interest.

In some examples, the operations may further include: processing the first image frame to generate a white light image; processing the second image frame to generate a fluorescence image; and providing the white light image and the fluorescence image to a display device communicatively coupled to the computing system for simultaneous display.

In other examples, the operations may further include: processing the first image frame to generate a white light image; processing the second image frame to generate a fluorescence image; blending the white light image and the fluorescence image to generate a combined image; and providing the combined image to a display device communicatively coupled to the computing system for display.

In further examples, the operations may further include: processing the first image frame to generate a white light image; identifying a boundary of the region of interest based on the comparing of the shifts in the sum of amplitudes across the plurality of second tiles; smoothing the boundary; and overlaying the boundary on the white light image to generate an annotated white light image.

According to further aspects, the techniques described herein relate to a method performed by a computing system communicatively coupled to a medical device, the method including: receiving a first image frame captured by an imaging assembly of the medical device as a first light source is emitting white light onto a target area; receiving a second image frame captured by the imaging assembly as a second light source is emitting ultraviolet light onto the target area to excite any fluorescent molecules present within the target area that are indicative of a region of interest; dividing the first image frame into a plurality of first tiles and the second image frame into a plurality of second tiles; determining a first intensity profile across the plurality of first tiles and a second intensity profile across the plurality of second tiles; comparing shifts in the second intensity profile to identify a subset of the plurality of second tiles corresponding to the region of interest; and using shifts in the first intensity profile as a control to confirm the subset of the plurality of second tiles correspond to the region of interest.

It may be understood that both the foregoing general description and the following detailed description are exemplary and explanatory only and are not restrictive of the invention, as claimed. As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate examples of this disclosure and, together with the description, serve to explain the principles of the disclosure.

FIG. 1 depicts an example environment in which a dual light imaging system may be implemented.

FIG. 2 depicts an example method for dual light image processing.

FIG. 3 depicts an example method for identifying regions of interest using dual light imaging.

FIG. 4 is a graph depicting example wavelength sensitivities of an image sensor of the dual light imaging system.

FIG. 5 is a graph depicting an example emission of a narrow band light source and a shifted re-emission spectrum.

FIG. 6 is a graph depicting an example timing for interchanging between white light and narrow band light source emissions in a hybrid operating mode of the dual light imaging system.

FIG. 7 is a graph depicting another example timing for interchanging between white light and narrow band light source emissions in the hybrid operating mode of the dual light imaging system.

FIGS. 8A-8C depict example images generated using the method of FIG. 2.

FIG. 9 depicts an example computing device.

### DETAILED DESCRIPTION

As briefly mentioned above, white light imaging performed using a medical imaging system, such as an endoscope, may allow a physician to visually identify and examine potential regions of interest, including regions of potentially cancerous tissue. Depending on the type and/or extent of cancerous tissue found, endoscopic-guided procedures may be used to resect or remove the affected regions. However, identifying a full extent of the potentially cancerous tissue and/or identifying margins to confirm that the area of tissue immediately surrounding the resected regions is healthy tissue (e.g., non-cancerous) can be challenging using only white light imaging.

Cancerous tissue may fluoresce or otherwise appear differently from healthy tissue natively and/or after exposure to dyes or liquids with fluorescent properties when excited with particular wavelengths of light, such as ultraviolet (UV) light. For example, certain types of cancerous tissues may have native fluorescence, and auto-fluoresce when excited. As another example, fluorescent dyes, when delivered to the target area, may bind to cancerous tissue and fluoresce when excited to identify the cancerous tissue. As a further example, after a particular liquid is ingested, proteins accumulated in cancerous tissue that have metabolized the liquid may fluoresce when excited to identify the cancerous tissue. Accordingly, performing fluorescence imaging in combination with white light imaging during an endoscopic procedure may provide further information about the tissue to help facilitate identification of regions of interest and/or determine clean resection margins, among other examples. Therefore, aspects of this disclosure are directed to systems, devices, and methods for performing dual light imaging, including white light imaging and fluorescence imaging.

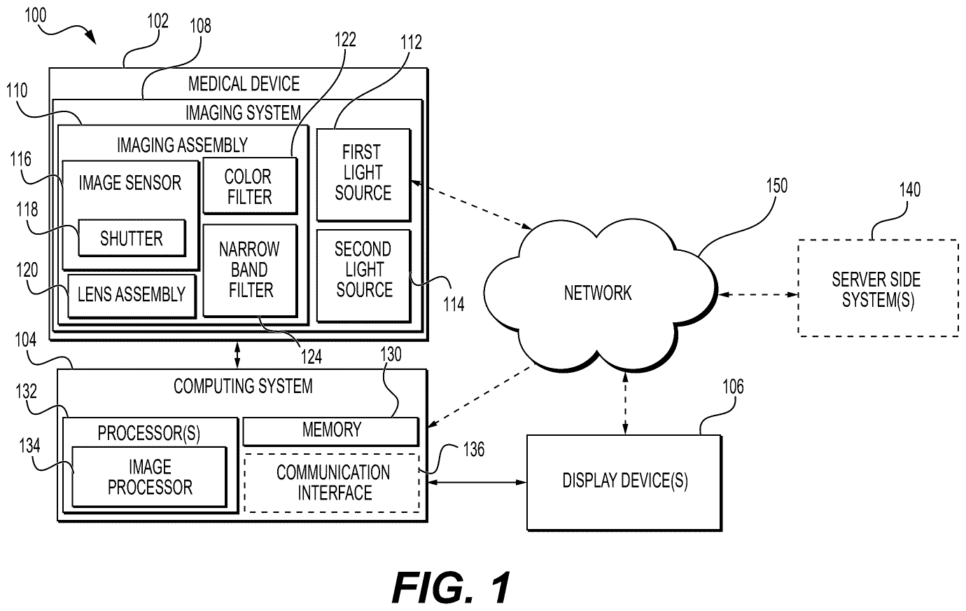

FIG. 1 depicts an exemplary environment 100 in which a dual light imaging system may be implemented. Environment 100 may include one or more of a medical device 102, a computing system 104, one or more display device(s) 106, one or more optional server side system(s) 140, and/or a network 150.

Medical device 102 may be used to perform a diagnostic and/or interventional medical procedure on a patient, hereinafter referred to as a medical procedure for brevity. Medical device 102 may be an endoscope or other type of scope, such as a bronchoscope, ureteroscope, duodenoscope, gastroscope, endoscopic ultrasonography (“EUS”) scope, colonoscope, laparoscope, arthroscope, cystoscope, aspiration scope, sheath, or catheter, among other examples.

Medical device 102 may include an imaging system 108. Imaging system 108 may include at least one imaging assembly 110, a first light source 112, and a second light source 114. In some examples, one or more components of imaging assembly 110, first light source 112, and/or second light source 114 may be located at a distal end of medical device 102 (e.g., at a distal tip of medical device 102). In other examples, one or more components of imaging assembly 110, first light source 112, and/or second light source 114 may be located at a proximal end of medical device 102 (e.g., at a handle of medical device 102). In further examples, first light source 112 and/or second light source 114 may instead be components of computing system 104.

Imaging assembly 110 may be configured to continuously capture image signals as the distal end of medical device 102 is inserted into and navigated through a body lumen of the patient to a target site or area during the medical procedure. First light source 112 and/or second light source 114 may be configured to illuminate areas of the patient's body, including the target area, during the medical procedure to facilitate different types of light imaging by imaging assembly 110. First light source 112 and second light source 114 may include one or more LEDs, incandescent light sources, optical fibers, and/or other illuminators that are each configured to emit light in a particular range of wavelengths. For example, first light source 112 may be a white light source configured to emit visible light, including wavelengths of light in a range of about 380-740 nanometers (nm), to enable white light imaging. Second light source 114 may be a narrow band light source configured to emit light in a narrower range of wavelengths to enable fluorescence imaging, for example, among other types of imaging that may use narrow bands of light wavelengths to achieve desired imaging results. In some examples, including the illustrative example below, the range of wavelengths of light emitted by first light source 112 and second light source 114 do not overlap (although they may closely border one another). However, in other examples, the range of wavelengths of light emitted by first light source 112 and second light source 114 may overlap.

To provide an illustrative example, to perform fluorescence imaging, second light source 114 may be a UV light source configured to emit wavelengths of light in a range of about 300 nm to about 370 nm. Wavelengths emitted by second light source 114 may be referred to as excitation wavelengths or excitation light based on their ability to excite any fluorescent molecules in the target area (e.g., to excite natively fluorescent cancerous tissues, dyes bound to cancerous tissues, and/or liquids metabolized by proteins of cancerous tissues). Upon excitement of a fluorescent molecule via the excitation light emitted by second light source 114, the fluorescent molecule decays, and the excitation light is absorbed and re-emitted back towards imaging system 108 in a red-shifted spectrum. For example, the re-emitted light has a longer wavelength, and is typically significantly less intense, than the excitation light. On the other hand, when the excitation light emitted by second light source 114 is directed toward a target area having no fluorescence molecules (e.g., an area of healthy tissue), the excitation light is scattered back towards imaging system 108 in a same or similar wavelength region.

Imaging system 108 may be configured to operate in at least two distinct modes, including a white light imaging mode and a fluorescence imaging mode. In the white light imaging mode, only first light source 112 may be powered on and controlled (e.g., via control signals sent via computing system 104) to emit white light for obtaining white light images of the target area. In the fluorescence imaging mode, only second light source 114 may be powered on and controlled (e.g., via control signals sent via computing system 104) to emit excitation light for obtaining fluorescence images of the target area. Additionally or alternatively, imaging system 108 may be configured to operate in a dual or hybrid imaging mode, such that both white light imaging and fluorescence imaging may be performed in parallel by interchanging or switching between emissions of white light and narrow band light, as described in more detail below.

Imaging assembly 110 may be a camera including an image sensor 116, a lens assembly 120, and one or more filters, such as a color filter 122 and a narrow band filter 124. Image sensor 116 may be a Complementary Metal Oxide Semiconductor (CMOS) sensor configured to convert light detected at image sensor 116 into image signals. For example, image sensor 116 includes a plurality of pixels (e.g., a grid of pixels) that convert detected photons to electrons. The signal charge generated from the electrons is converted into an electrical signal (e.g., a voltage), which may be further converted to a digital value using an Analog to Digital Converter (ADC), for example. The image signals are then provided to computing system 104 for processing into images. Generally, image sensor 116 may be configured to detect visible or white light.

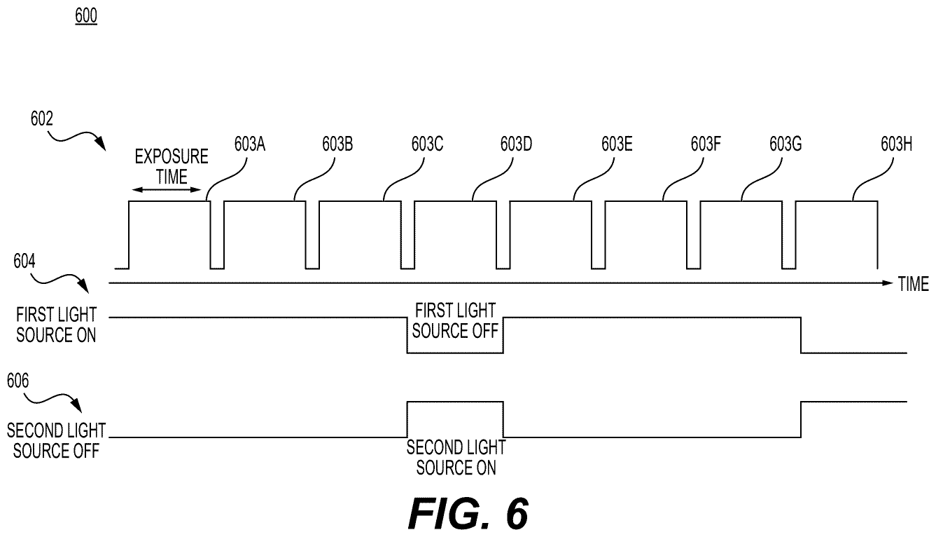

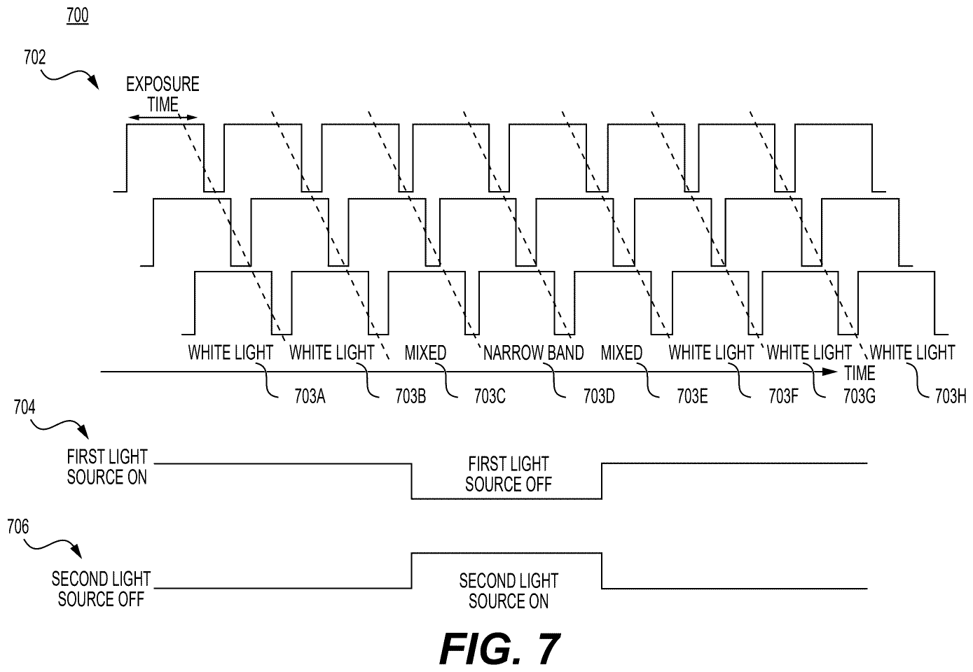

Lens assembly 120 includes one or more lenses configured to focus the light onto and control an amount of the light that enters image sensor 116. Sensor 116 includes a shutter 118. Shutter 118 is configured to control a length of time that the light is permitted to pass through lens assembly 120 to image sensor 116 (e.g., control an exposure time). In some examples, shutter 118 is a global shutter. When shutter 118 is a global shutter, all of the pixels of image sensor 116 are read out simultaneously upon exposure to the light for a single image frame. In other examples, shutter 118 is a rolling shutter. When shutter 118 is a rolling shutter, the pixels of image sensor 116 are read out row by row upon exposure to the light. This results in a delay. For example, as a first row of a current image frame is being exposed to light, a last row of a previous image frame is still being read out. Therefore, and as described in more detail below with reference to FIGS. 6 and 7, computing system 104 may perform different image processing techniques dependent on a type of shutter 118 included in imaging assembly 110.

Color filter 122 is a color filter array or color filter mosaic placed over the grid of pixels of image sensor 116 to capture information about a color of the light detected by image sensor 116 to enable generation of full-color images. For example, color filter 122 may be a Bayer filter configured to capture an intensity of light in red, green, and blue wavelength regions.

Narrow band filter 124 may be a filter configured to block a narrow range of wavelengths corresponding to the range of excitation wavelengths emitted by second light source 114. Continuing the illustrative example above, when second light source 114 is the UV light source configured to emit excitation wavelengths in a range of about 300 nm to about 370 nm, narrow band filter 124 is configured to at least block wavelengths of light in the range about 300 nm to about 370 nm. The range of excitation wavelengths emitted by second light source 114 are blocked by narrow band filter 124 to avoid image saturation. For example, the narrow band filter 124 helps to prevent image sensor 116 from detecting any of the emitted excitation wavelengths that are scattered off of healthy tissue within the target area back toward imaging system 108 in the same or similar wavelength region. However, in instances where fluorescent molecules are present within the target area (e.g., indicative of abnormal tissue), because the excitation light is absorbed and re-emitted by fluorescent molecules in a red-shifted spectrum that falls outside of the narrow range of wavelengths blocked by narrow band filter 124, the re-emitted light is detectable by image sensor 116 to enable identification of a region including the fluorescent molecules as a region of interest.

In some embodiments of imaging system 108, narrow band filter 124 may be omitted. For example, narrow band filter 124 may be omitted when the excitation wavelengths emitted by second light source 114 fall outside a spectrum detectable by image sensor 116 (e.g., fall outside the visible light spectrum). In other examples, the narrow band filter 124 may be included even when the excitation wavelengths emitted by second light source 114 fall outside the spectrum detectable by image sensor 116, and particularly when the range of excitation wavelengths closely borders the detectable spectrum.

One or more components of medical device 102, including imaging system 108 and the components thereof, may be communicatively coupled to computing system 104 via wired connections and/or wireless connections (e.g., over network 150) to enable communication of various signals between medical device 102 and computing system 104. For example, the image signals generated by imaging assembly 110 may be received by computing system 104 for processing. Additionally, computing system 104 may provide one or more control signals to the imaging assembly 110, first light source 112, and/or second light source 114 to cause and/or modify an operation thereof (e.g., to power on or off, to adjust operating parameters, etc.).

In some examples, computing system 104 is a controller, a control unit, a computing device, or other similar standalone processing unit separate from medical device 102. In other examples, computing system 104 may be integrated with medical device 102. For example, computing system 104 may be positioned in a handle of medical device 102. In other examples, computing system 104 may be positioned at the distal end of medical device 102.

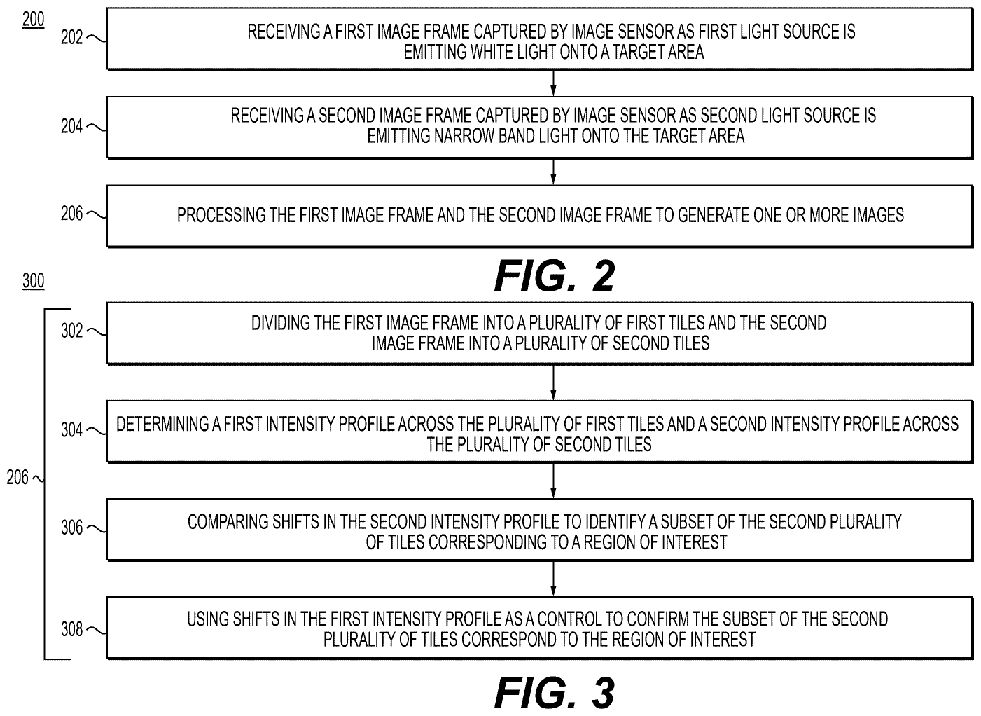

Computing system 104 may include a memory 130 and one or more processor(s) 132. Memory 130 may store instructions to be executed by processor(s) 132 to cause computing system 104 to perform corresponding operations. At least a portion of the instructions stored in memory 130 may include one or more imaging processes, including dual light imaging processes. Memory 130 may also include one or more data stores. Additionally or alternatively, computing system 104 may include one or more data stores separate from memory 130. Processor(s) 132 may include at least one image processor 134. Image processor 134 may be configured to process one or more image signals received from and captured by imaging assembly 110 when white light and/or narrow band light is being emitted by first light source 112 and/or second light source 114, respectively, as described in detail with reference to FIGS. 2 and 3 below. In some examples, image processor 134 may be or include a field-programmable gate array (FPGA), a digital signal processing (DSP) processor, a graphics processing unit (GPU), or the like.

Computing system 104 may further include an optional communication interface 136 for providing connectivity to network 150. Optional communication interface 136 may also provide connectivity to medical device 102 and/or display device(s) 106. In some examples, a communicative connection between computing system 104 and medical device 102 (or components thereof) and/or computing system 104 and display device(s) 106 may be at least partially supported via network 150.

Display device(s) 106 may be configured to display image data, including at least the images generated by computing system 104. Display device(s) 106 may include one or more of a combination of monitors, computing device screens, touch screen display devices, etc. In some examples, one or more of the display device(s) 106 may be a separate device from computing system 104 that is communicatively coupleable to computing system 104 via wired and/or wireless connections. In other examples, at least one of display device(s) 106 may be a display of computing system 104 itself.

In some examples, computing system 104 may generate, or may cause to be generated, one or more graphical user interfaces based on instructions or information stored in memory 130, instructions or information received from one or more optional server side system(s) 140, and/or the like and may cause the graphical user interfaces to be displayed via display device(s) 106. The graphical user interfaces may be, for example, application interfaces or browser user interfaces and may include text, selection controls, and/or the like, in addition to the displayed image data. Display device(s) 106 may include a touch screen or a display with other input systems (e.g., a mouse, keyboard, voice, etc.) for an operator of computing system 104 to control functions of computing system 104, medical device 102 (or components thereof) via computing system 104, and/or display device(s) 106.

As one example, the operator may select one or more of the control elements displayed on a graphical user interface of display device(s) 106 to select an imaging mode for imaging system 108 (e.g., select the white light imaging mode, the fluorescence imaging mode, or the dual or hybrid imaging mode). The selection may be received by computing system 104 and cause corresponding signals to be transmitted from computing system 104 to imaging system 108 and/or specific components thereof. In some examples, additional control elements may be displayed on a graphical user interface of display device(s) 106 that enable adjustment of a frequency or duration of the fluorescence imaging. For example, based on fluorescence lifetime, a longer duration under fluorescence imaging may be desired to obtain more fluorescence image frames (e.g., more samples), and mitigate potential dilution to the samples caused by interchanged white light imaging to enable more accurate identification of regions of interest. Additionally, an increase in an exposure time for the fluorescence image frames (e.g., by increasing the duration of time that the light is permitted to pass through lens assembly 120 to image sensor 116 via shutter 118) may help to provide enhanced images.

One or more components of environment 100, such as medical device 102, computing system 104, and/or display device(s) 106, may be capable of network connectivity, and may communicate with one another over a wired or wireless network, such as network 150. Network 150 may be an electronic network. Network 150 may include one or more wired and/or wireless networks, such as a wide area network (“WAN”), a local area network (“LAN”), personal area network (“PAN”), a cellular network (e.g., a 3G network, a 4G network, a 5G network, etc.), or the like. In other examples, the components of environment 100 may communicate and/or connect to network 150 over universal serial bus (USB) or other similar local, low latency connections or direct wireless protocol. Components of environment 100 may be connected via network 150, using one or more standard communication protocols, such that the component may transmit and receive communications from each other across network 150.

In some examples, when one or more of the components of environment 100 are capable of connecting to network 150, environment 100 may also include one or more optional server side system(s) 140. Optional server side system(s) 140 may include one or more of remote image processing systems configured to perform at least a portion of the image processing, including but not limited, more resource intensive processes, such as machine learning processes (e.g., to conserve local resources of computing system 104 when network connectivity is available). Additionally or alternatively, optional server side system(s) 140 may include data storage systems for storing the image generated by computing system 104 (e.g., in response to receiving an action input from the operator to record or otherwise save the image). In some examples, at least one of the data storage systems may include a picture archiving and communication system (PACS) that stores the image, along with other types of imaging data from various imaging modalities (e.g., ultrasound, magnetic resonance, nuclear medicine imaging, positron emission tomography, computed tomography, mammograms, digital radiography, histopathology, etc.) associated with the patient.

Although various components in environment 100 are depicted as separate components in FIG. 1, it should be understood that a component or portion of a component in environment 100 may, in some embodiments, be integrated with or incorporated into one or more other components. As one example, first light source 112 and/or second light source 114 may instead be integrated components of computing system 104. As another example, one of display device(s) 106 may be integrated with computing system 104 and/or computing system 104 may be integrated with medical device 102. In some embodiments, operations or aspects of one or more of the components discussed above may be distributed amongst one or more other components. Any suitable arrangement and/or integration of the various systems and devices of environment 100 may be used.

The specific examples included throughout this disclosure implement an endoscopic imaging system configured to perform, in real or near real-time during a medical procedure, dual white light and fluorescence imaging. However, it should be understood that techniques according to this disclosure may be adapted to other medical imaging systems having varying types of imaging devices and narrow band light sources enabling other imaging modalities. For example, rather than analyzing a fluorescence profile to identify regions of interest, absorption and reflection/scattering behavior at varying wavelengths of light could be analyzed to identify regions of interest. It should also be understood that the examples above are illustrative only. The techniques and technologies of this disclosure may be adapted to any suitable activity.

FIG. 2 depicts an example method 200 for dual light image processing. In some examples, one or more steps of method 200 may be performed by computing system 104. At step 202, method 200 may include receiving a first image frame captured by image sensor 116 as first light source 112 is emitting white light onto a target area. In some examples, the first image frame may be captured when imaging system 108 is operating in a white light imaging mode. In other examples, the first image frame may be captured when imaging system 108 is operating in the dual or hybrid imaging mode and first light source 112 is powered on and controlled by computing system 104 to emit white light. Additionally, for simplicity, step 202 describes the receiving of one first image frame. However, a plurality of first image frames may be sequentially or interchangeably received dependent on the operating mode of imaging system 108.

At step 204, method 200 may include receiving a second image frame captured by image sensor 116 as second light source 114 is emitting narrow band light onto the target area, such as UV light to excite any fluorescent molecules indicative of abnormal or cancerous tissue in the target area. In some examples, the second image frame may be captured after imaging system 108 is switched from operating in the white light imaging mode to the fluorescence imaging mode. In other examples, the second image frame may be captured when imaging system 108 is operating in the dual or hybrid imaging mode and second light source 114 is powered on and controlled by computing system 104 to emit narrow band light. Additionally, for simplicity, step 204 describes the receiving of one second image frame. However, a plurality of second image frames may be sequentially or interchangeably received dependent on the operating mode of imaging system 108.

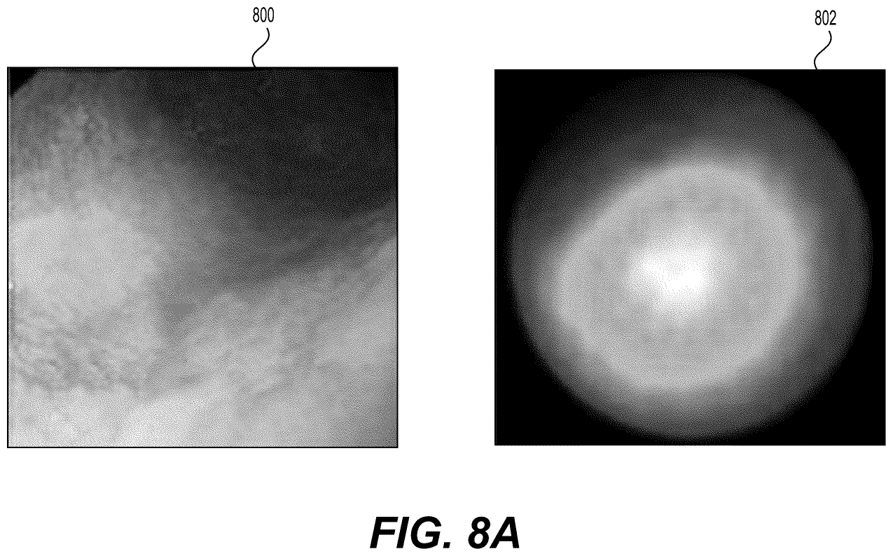

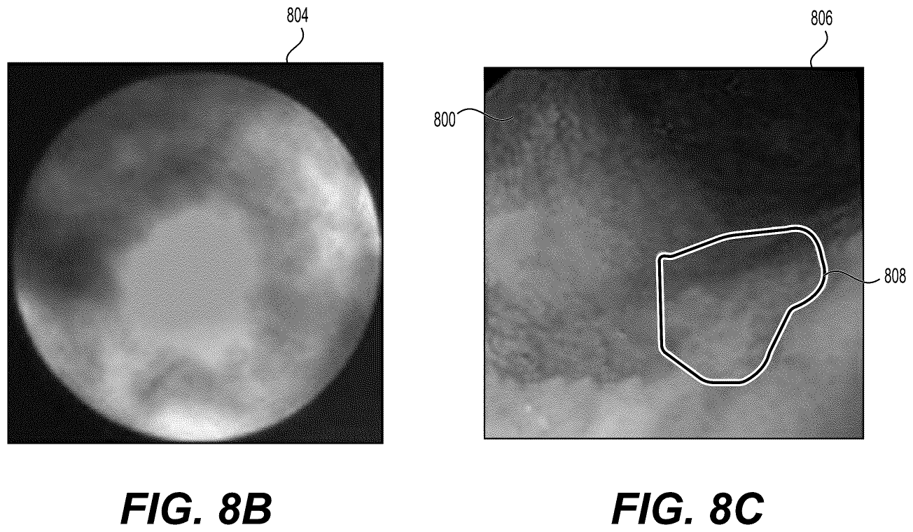

At step 206, method 200 may include processing the first image frame and the second image frame to generate one or more images. In some examples, the images may be provided to one of device(s) 106 for display. Example types of images that may be generated and/or displayed include a white light image, a fluorescent image, a combined image overlaying the fluorescent image on the white light image, and/or an annotated white light image including information extracted from the fluorescent image, addressed in turn below and as shown in FIGS. 8A-8C.

In some examples, and as described in detail in below with reference to FIG. 3, additional processing techniques may be applied to the first and second image frames to determine intensity profiles across the first and second image frames for use in identifying one or more regions of interest. Such identification of regions of interest may be used to facilitate generation of one or more types of the images, and/or generate other types of output (e.g., notifications, alerts, etc.).

The first image frame may be processed to generate a white light image of the target area. In examples where imaging system 108 includes narrow band filter 124, and/or any portion of the range of wavelengths blocked by narrow band filter 124 falls within or is in close proximity to the range of detection of image sensor 116, a color of the white light image may be affected. Although any effects would be negligible given the narrow range of wavelengths blocked, color correction techniques may be applied to mitigate or eliminate the effects.

In examples where the first image frame is received when imaging system 108 is operating in the white light imaging mode, the first image frame may be processed prior to receiving the second image frame at step 204. For example, the white light image may be generated and displayed (e.g., via device(s) 106). An operator, such as a physician, may visually observe a potential region of interest that prompts the operator to interact with computing system 104 to cause the imaging system 108 to switch from white light imaging mode to either the fluorescence imaging mode or the dual or hybrid imaging mode to enable capture of the second image frame.

The second image frame may be processed to generate a fluorescence image. In the absence of fluorescence molecules within the target area, the fluorescence image generated will be dark (e.g., because narrow band filter 124 blocked any emitted excitation light that was scattered back off of healthy tissue at the same or similar wavelength from being detected by image sensor 116). However, if one or more fluorescence molecules are present, absorption of the excitation light by the fluorescence molecules will result in re-emission of the light at the red-shifted wavelength (e.g., a wavelength outside of the band that is blocked by the narrow band filter 124). The resulting spectrum of light detected by image sensor 116 will include the re-emitted light, and the generated image will depict the location of the fluorescence molecules. The fluorescence image can be displayed separately or simultaneously (e.g., in a dual side-by-side view) with the white light image, for example, on display device(s) 106.

In some examples, the white light image and the fluorescence image may be further processed to generate a combined image. As one example, the white light image and the fluorescence image may be blended with one another such that the fluorescence image is, for example, overlaid on the white light image. In such examples, optional color mapping may be performed to change a color of the combined image to make the fluorescence depicted in the overlaid fluorescence image more visible in the combined image. For example, if the fluorescence depicted is green, color mapping may be applied to change the fluorescence from green to red.

In further examples, a boundary of a region of interest identified via additional processing of at least the fluorescence image may be smoothed and overlaid on the white light image to generate an annotated white light image. For example, and as described in more detail with reference to FIG. 3, a pixel intensity profile of the fluorescence image frame (e.g., a fluorescence profile) may be determined and used to identify the boundary. For example, the boundary may be identified at locations where there is a change or difference in neighboring pixel intensity above a predefined threshold. In other examples, alternative techniques such as edge detection (e.g., binary edge detection, Sobel filter application, etc.) may be used to identify the boundary.

Accordingly, certain aspects may include dual light image processing. Method 200 described above is provided merely as an example, and may include additional, fewer, different, or differently arranged steps than depicted in FIG. 2.

FIG. 3 depicts an example method 300 for identifying regions of interest using dual light imaging. In some examples, one or more steps of method 300 may be performed by computing system 104. Method 300 may be one example processing technique performed as part of step 206 of method 200 described with reference to FIG. 2. In some examples, method 300 may be performed on raw image data (e.g., on the first and second image frames received at steps 202 and 204 of method 200). In other examples, the method 300 may be performed after color processing of the first and second image frames.

At step 302, method 300 may include dividing the first image frame into a plurality of first tiles and the second image frame into a plurality of second tiles. Each of the plurality of first tiles and second tiles may include a predetermined number of pixels.

At step 304, method 300 includes determining a first intensity profile across the plurality of first tiles and a second intensity profile across the plurality of second tiles. Determining the first and second intensity profiles includes, for each tile of the plurality of first tiles and the plurality of second tiles, determining a sum of amplitudes for first, second, and third pixels within the respective tile. For example, the predetermined number of pixels included within a respective tile may include one or more first pixels (e.g., red pixels), one or more second pixels (e.g., blue pixels), and one or more third pixels (e.g., green pixels). An amplitude value representing an intensity of each of the one of more first pixels is determined and summed to yield a first sum of amplitudes for the first pixels. An amplitude value representing an intensity of each of the one of more second pixels is determined and summed to yield a second sum of amplitudes for the second pixels. An amplitude value representing an intensity of each of the one of more third pixels is determined and summed to yield a third sum of amplitudes for the third pixels. The first, second, and third sums determined for each of the plurality of first tiles and the plurality of second tiles generate a simple spectrum of light (e.g., the first and second intensity profiles) detected by image sensor 116.

As previously mentioned, method 300 may be performed on raw image data of the first and second image frames or post-color processing. However, performing method 300 on the raw image data may help to enable the absolute amplitude values of each pixel to be determined as opposed to color processed data, which includes amplitudes from neighboring pixels embedded in the each pixel (e.g., altering the amplitude value for the respective pixel).

At step 306, method 300 includes comparing shifts in the second intensity profile to identify a subset of the plurality of second tiles corresponding to a region of interest. Comparing the shifts in the second intensity profile includes comparing shifts in the sum of amplitudes across the plurality of second tiles. In one example, a sliding window may be used to compare the first sum of amplitudes for the first pixels, the second sum of amplitudes for the second pixels, and the third sum of amplitudes for the third pixels between or among neighboring tiles of the plurality of second tiles to identify differences or changes in the first, second, and/or third sums. In some examples, the differences may further be compared to a predefined threshold. Such differences or changes, and particularly differences or changes exceeding the predefined threshold, may indicate that the tiles correspond to a border between healthy tissue and potentially cancerous tissue. Therefore, the subset of the plurality of second tiles having shifts in the first, second, and/or third sums above the predefined threshold are indicative of boundaries of potential regions of interest.

To provide an illustrative example, excitation from a UV light source around 370 nm produces a fluorescence in approximately the 450 nm to 500 nm range of different human tissue. The intensity of the fluorescence can change significantly from healthy tissue to cancerous tissue, including near the border between the two. For example, if a UV light source is emitted onto a target area including fluorescence molecules that fluoresce in the green color channel, green pixels of the tiles corresponding to a location of the fluorescent molecules in the second image frame will exhibit an increased amplitude value (e.g., an increased intensity).

At step 308, method 300 may include using shifts in the first intensity profile as a control to confirm the subset of the plurality of second tiles correspond to the region of interest. The shifts in the first intensity profile may be shifts in the sum of amplitudes across the plurality of first tiles. For example, a sliding window may be used to compare the first sum of amplitudes for the first pixels, the second sum of amplitudes for the second pixels, and the third sum of amplitudes for the third pixels between or among neighboring tiles of the plurality of first tiles to identify differences or changes in the first, second, and/or third sums. The plurality of first tiles correspond to the plurality of second tiles, and the differences identified from the plurality of first tiles may be subtracted from the differences identified from the corresponding plurality of second tiles. The subtraction helps to ensure that the differences identified from the plurality of second tiles are a result of different fluorescent properties of healthy versus abnormal or cancerous tissue, as opposed to a natural change in scene, such as a shadow created by contours of the tissue.

Method 300 describes processing of each of the first and second image frames to enable the first image frame to act as a control when identifying regions of interest. However, in other examples, only the second image frame may be processed according to steps 302-306 to identify regions of interest. Accordingly, certain aspects may include dual light image processing. Method 300 described above is provided merely as an example, and may include additional, fewer, different, or differently arranged steps than depicted in FIG. 3.

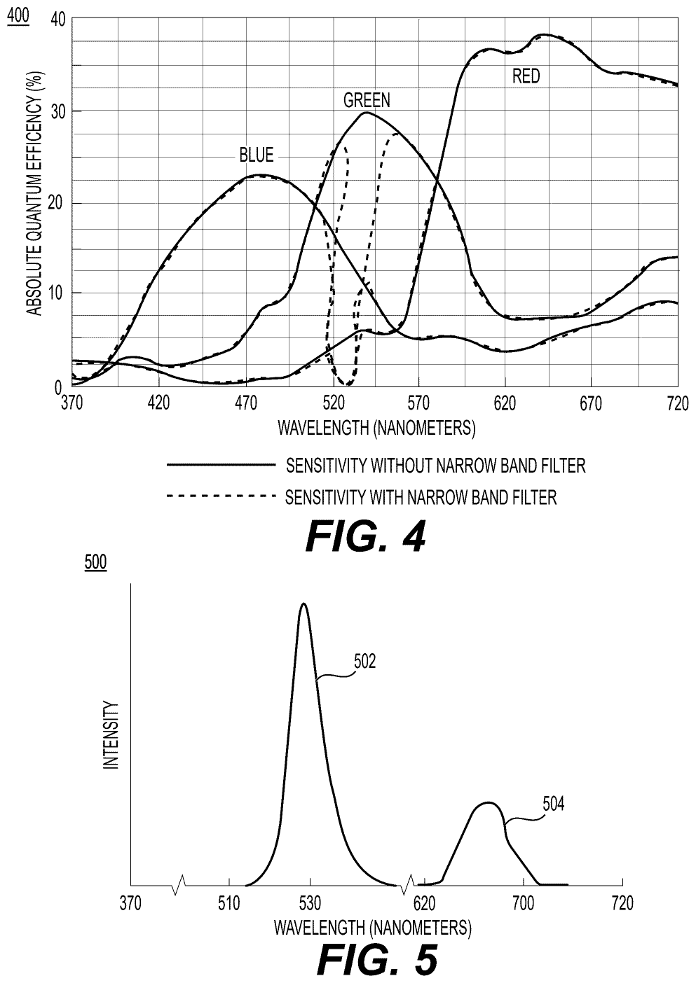

FIG. 4 is a graph 400 depicting example wavelength sensitivities of an image sensor of a dual light imaging system, such as image sensor 116 of imaging system 108. X-axis of graph 400 represents wavelength along the visible light spectrum, and y-axis of graph 400 represents a percentage of absolute quantum efficiency (e.g., ratio of light emitted to light absorbed) used as measure of pixel response. Wavelength sensitivity, as depicted by graph 400, includes a pixel response for each of first, second, and third pixels of image sensor 116 (e.g., red, green, and blue pixels enabled by color filter 122) at each wavelength along the visible light spectrum. The solid lines depict the wavelength sensitivity of image sensor 116 when narrow band filter 124 is omitted from imaging system 108. The dashed lines represent the wavelength sensitivity of image sensor 116 when imaging system 108 includes narrow band filter 124. Specifically, when narrow band filter 124 is included, absolute quantum efficiency (e.g., absorption) is reduced close to about 0% around a peak of narrow band filter 124. For example, as shown in FIG. 4, the peak of narrow band filter may be about 520 nm to about 530 nm.

FIG. 5 is a graph 500 depicting an example emission 502 of a narrow band light source, such as second light source 114, and a shifted re-emission spectrum 504. X-axis of graph 500 represents wavelength of light along the visible light spectrum, and y-axis of graph 500 represents an intensity of the light. When imaging system 108 includes narrow band filter 124, and emission 502 of narrow band light is performed by second light source 114, wavelengths in the range of emission 502 are blocked by narrow band filter 124. Such blocking helps to prevent image sensor 116 from detecting wavelengths from emission 502 that are scattered off of healthy tissue back toward imaging system 108 in the same or similar wavelength region. Therefore, if no fluorescent molecules are present in the area onto which emission 502 was directed, the wavelengths scattered back to imaging system 108 will be blocked by narrow band filter 124, and a dark fluorescence image will result. However, if any fluorescent molecules are present in the area onto which emission 502 was directed, wavelengths from emission 502 will be absorbed and re-emitted by the fluorescent molecules in shifted re-emission spectrum 504. Light within shifted re-emission spectrum 504 has a longer wavelength (e.g., is red-shifted), and is of lesser intensity than light of emission 502. Shifted re-emission spectrum 504 falls outside of the narrow range of wavelengths blocked by narrow band filter 124, but within the sensitivity of image sensor 116. Therefore, shifted re-emission spectrum 504 is detectable by image sensor 116 to enable identification of regions of interest indicated by the fluorescent molecules.

FIG. 6 is a diagram 600 depicting an example timing for interchanging between white light and narrow band light source emissions in a hybrid operating mode of a dual light imaging system, such as imaging system 108 having first light source 112 and second light source 114. This example timing may be implemented in instances where shutter 118 of imaging assembly 110 is a global shutter. Shutter 118 is configured to control a duration of time that the light is permitted to pass through lens assembly 120 to image sensor 116 (e.g., control an exposure time). Each exposure to light results in an image frame that is generated by image sensor 116. When shutter 118 is a global shutter, all of the pixels of image sensor 116 are read out simultaneously for the image frame. Therefore, switching between first light source 112 and second light source 114 at predefined intervals corresponding to a duration or period of image frame exposure enables an entirety of the image frame to be exposed with one type of light source. For example, every Nth image frame may be illuminated with second light source 114 instead of first light source 112 and processed separately (e.g., without having to do any further extractions to identify the Nth frame in contrast from the process described with reference to FIG. 7 below).

To provide an illustrative example, a first portion 602 of diagram 600 represents a plurality of periods of exposure time and a corresponding plurality of image frames 603A-H captured as imaging system 108 is interchanging between white light and narrow band emissions from first light source 112 and second light source 114 in the hybrid operating mode. A second portion 604 of diagram 600 represents a power state (e.g., on or off) of first light source 112 throughout the periods of exposure time. A third portion 606 of diagram 600 represents a power state (e.g., on or off) of second light source 114 throughout the periods of exposure time. As illustrated, only one of first light source 112 and second light source 114 is powered on at a given time in the hybrid operating mode.

Initially, first light source 112 may be powered on by computing system 104, such that white light is emitted onto the target area while a first image frame 603A, a second image frame 603B, and a third image frame 603C (e.g., white light image frames) are captured. Next, first light source 112 may be powered off, and second light source 114 may be powered on by computing system 104, such that narrow band light is emitted onto the target area while a fourth image frame 603D (e.g., a fluorescence image frame) is captured. Then, second light source 114 may be powered off, and first light source 112 powered back on, and the same interchange or alternation pattern may continue. For example, a fifth image frame 603E, a sixth image frame 603F, and a seventh image frame 603G (e.g., white light image frames) may be captured while first light source 112 is powered on and emitting white light onto the target area, followed by a capture of an eight image frame 603H (e.g., a fluorescence image frame) while second light source 114 is powered on and emitting narrow band light onto the target area.

A white light emission followed by a narrow band light emission may represent one cycle in the interchange or alternation pattern. While two cycles of the interchange or alternation pattern between the white light and narrow band light source emissions are depicted in FIG. 6, more or fewer cycles may be performed. For example, the cycle may be repeated a plurality of times to enable sufficient samples (e.g., image frames) to be collected and processed for visualization and/or assessment of the target area. Additionally, in the cycle depicted in FIG. 6, every fourth image frame is illuminated with second light source 114. However, in other examples, different image frame intervals may be utilized for the interchange or alternation pattern.

FIG. 7 is a diagram 700 depicting another example timing for interchanging between white light and narrow band light source emissions in a hybrid operating mode of a dual light imaging system, such as imaging system 108 having first light source 112 and second light source 114. This other example timing may be implemented in instances where shutter 118 of imaging assembly 110 is a rolling shutter. When shutter 118 is a rolling shutter, the pixels of image sensor 116 are read out row by row upon exposure to the light. This results in a delay. For example, as a first row of a current image frame is being exposed to light, a last row of a previous image frame is still being read out. Therefore, whenever there is a switch between white light and narrow band light emissions (e.g., when first light source 112 is powered off and second light source 114 is powered on or vice versa) a mixed image frame is captured.

To provide an illustrative example, a first portion 702 of diagram 700 represents a plurality of periods of exposure time and a plurality of image frames 703A-H (the diagonal lines depicting the readout delay) captured as imaging system 108 is interchanging between white light and narrow band emissions from first light source 112 and second light source 114 in the hybrid operating mode. A second portion 704 of diagram 700 represents a power state (e.g., on or off) of first light source 112 throughout the periods of exposure time. A third portion 706 of diagram 700 represents a power state (e.g., on or off) of second light source 114 throughout the periods of exposure time. As illustrated, only one of first light source 112 and second light source 114 is powered on at a given time.

Initially, first light source 112 may be powered on by computing system 104, such that white light is emitted onto the target area while a first image frame 703A and a second image frame 703B (e.g., white light image frames) are captured. Additionally, a capture of a third image frame 603C may be initiated just prior to a light source switch.

Next, first light source 112 may be powered off, and second light source 114 may be powered on by computing system 104, such that narrow band light is emitted onto the target area to capture a fourth image frame 703D. Due to the rolling shutter readout delay, as a first row of fourth image frame 703D is being exposed to narrow band light after the switch (e.g., to generate a fluorescence image frame), a last row of third image frame 703C initially exposed to white light before the switch is still being read out. Additionally or alternatively, the last row (or other rows) of third image frame 703C may be exposed to both white light and narrow band light (e.g., based on timing of the switch). This results in third image frame 703C being a mixed image frame.

As narrow band light is continuing to be emitted by second light source 114, a capture of a fifth image frame 703E may be initiated just prior to a light source switch back. Again, due to the rolling shutter readout delay, when second light source 114 is powered off and first light source 112 is powered back on, as a first row of a sixth image frame 703F is being exposed to white light after the switch back, a last row of fifth image frame 703E initially exposed to narrow band light before the switch back is still being read out. This results in the fifth image frame 703E being a mixed image frame. Sixth image frame 703F, as well as a seventh image frame 703G and an eight image frame 703H, each captured after the light source switch back, may be white light images. A white light emission followed by a narrow band light emission may represent one cycle in the interchange or alternation pattern. While a first cycle and a portion of a second cycle of the interchange or alternation pattern between the white light and narrow band light source emissions are depicted in FIG. 7, more or fewer cycles may be performed. For example, the cycle may be repeated a plurality of times to enable sufficient samples (e.g., image frames) to be collected and processed for visualization and/or assessment of the target area.

In implementations where shutter 118 is a rolling shutter, the mixed image frames, such as third image frame 703C and fifth image frame 703E, are identified and discarded. The mixed image frames are discarded to help ensure further image processing, such as one or more of the image processing techniques described above with reference to FIGS. 2 and 3, is performed only on the white light image and fluorescence images to maintain accuracy. To identify the mixed image frames, a timing of a start of capture of the image frames is monitored relative to the power state of first light source 112 and the power state of second light source 114.

In either example where shutter 118 is a global shutter (FIG. 6) or is a rolling shutter (FIG. 7), a frame rate of the white light imaging may be reduced for the benefit of the fluorescence imaging. However, if spaced appropriately, the loss of individual frames from the white light imaging will not be noticeable by the user. For example, if imaging assembly 110 is running at 60 frames per second (fps), taking a fluorescence image once per second will result in 57 white light frames per second (e.g., above the required minimum of 25 frames fps for the observation of fluid motion). Depending on the particular implementation, the duration and frequency of the fluorescence imaging relative to white light imaging may further be adjustable by the user.

FIGS. 8A-8C depict example images generated using method 200 of FIG. 2. FIG. 8A shows an example dual view of a white light image 800 and a fluorescence image 802 to enable simultaneous but separate display (e.g., on one of display device(s) 106). FIG. 8B shows an example combined image 804 generated from a blending of white light image 800 and fluorescence image 802, for example. FIG. 8C shows an example annotated white light image 806, where a boundary 808 of a region of interest identified from fluorescence image 802 (e.g., identified using method 300 of FIG. 3) may be smoothed and overlaid on the white light image 800.

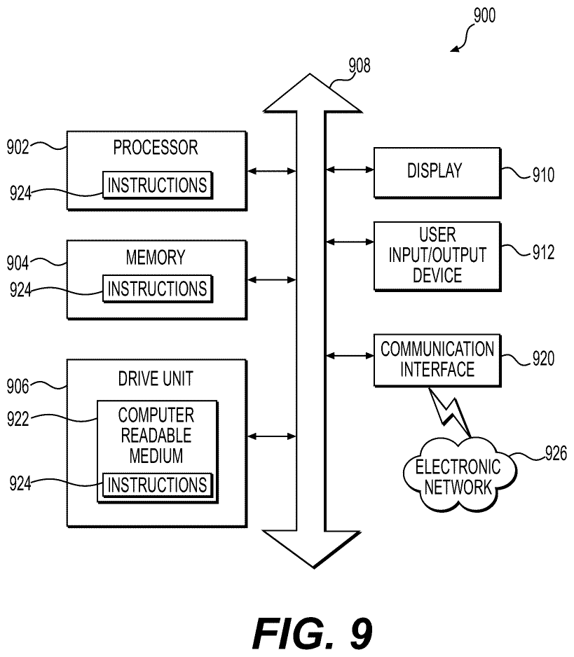

FIG. 9 depicts an example of a computer 900. FIG. 9 is a simplified functional block diagram of computer 900 that may be configured as a device for executing processes, steps, or operations depicted in, or described with respect to, FIGS. 2-8C and, according to exemplary embodiments of this disclosure. For example, computer 900 may be configured as one or more of medical device 102, computing system 104, display device(s) 106, optional server side system(s) 140, and/or another device or component according to exemplary embodiments of this disclosure. In various embodiments, any of the systems herein may be or include computer 900 including, for example, a data communication interface 920 for packet data communication. Computer 900 may communicate with one or more other computers, for example, using an electronic network 926 (e.g., via data communication interface 920). Electronic network 926 may include a wired or wireless network, for example, similar to network 150 depicted in FIG. 1.

Computer 900 also may include a central processing unit (“CPU”), in the form of one or more processors 902, for executing program instructions 924. Program instructions 924 may include at least instructions for performing image processing, including region of interest-based automatic brightness control (e.g., if computer 900 is computing system 104).

Computer 900 may include an internal communication bus 908. Computer 900 may also include a drive unit 906 (such as read-only memory (ROM), hard disk drive (HDD), solid-state disk drive (SDD), etc.) that may store data on a computer readable medium 922 (e.g., a non-transitory computer readable medium), although computer 900 may receive programming and data via network communications. Computer 900 may also have a memory 904 (such as random-access memory (RAM)) storing instructions 924 for executing techniques presented herein. It is noted, however, that in some aspects, instructions 924 may be stored temporarily or permanently within other modules of computer 900 (e.g., processor 902 and/or computer readable medium 922). Computer 900 also may include user input and output devices 912 and/or a display 910 to connect with input and/or output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may, at times, be communicated through the Internet or various other telecommunication networks. Such communications, for example, may enable loading of the software from one computer or processor into another. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A system for dual light imaging, the system comprising:
a medical device including an imaging assembly configured to capture image frames of a target area;
a first light source configured to emit white light;
a second light source configured to emit narrow band light; and
a computing system communicatively coupled to the imaging assembly, the first light source, and the second light source, and configured to perform operations including:
receiving a first image frame captured by the imaging assembly as the computing system controls the first light source to emit the white light onto the target area;
receiving a second image frame captured by the imaging assembly as the computing system controls the second light source to emit the narrow band light onto the target area;
dividing the first image frame into a plurality of first tiles and the second image frame into a plurality of second tiles;
determining a first intensity profile across the plurality of first tiles and a second intensity profile across the plurality of second tiles;
comparing shifts in the second intensity profile to identify a subset of the plurality of second tiles corresponding to a region of interest within the target area; and
using shifts in the first intensity profile as a control to confirm the subset of the plurality of second tiles correspond to the region of interest.

2. The system of claim 1, wherein determining the first intensity profile and the second intensity profile includes:
for each tile of the plurality of first tiles and the plurality of second tiles, determining a sum of amplitudes for first, second, and third pixels within the respective tile.

3. The system of claim 2, wherein comparing the shifts in the second intensity profile includes:
identifying differences above a predefined threshold in the sum of amplitudes across the plurality of second tiles.

4. The system of claim 3, wherein using the shifts in the first intensity profile as a control includes:
identifying and subtracting differences in the sum of amplitudes across the plurality of first tiles from the differences in the sum of amplitudes across the plurality of second tiles.

5. The system of claim 1, the operations further including:
processing the first image frame to generate a white light image; and
processing the second image frame to generate a fluorescence image.

6. The system of claim 5, the operations further including:
providing the white light image and the fluorescence image to a display device communicatively coupled to the computing system for simultaneous display.

7. The system of claim 5, the operations further including:
generating a combined image by blending the white light image and the fluorescence image; and
providing the combined image to a display device communicatively coupled to the computing system for display.

8. The system of claim 7, wherein the fluorescence image depicts fluorescence in a first color, and wherein the operations further include:
performing a color mapping to change the first color to a second color to increase visibility of the fluorescence in the combined image.

9. The system of claim 5, the operations further including:
identifying a boundary of the region of interest based on the comparing of the shifts in the second intensity profile;
smoothing the boundary; and
overlaying the boundary on the white light image to generate an annotated white light image.

10. The system of claim 1, wherein the imaging assembly includes a narrow band filter configured to block a range of wavelengths corresponding to the narrow band light emitted by the second light source from being detected by the imaging assembly.

11. The system of claim 10, wherein the second light source is a UV light source configured to emit the narrow band light to excite any fluorescent molecules within the target area, and wherein a re-emission spectrum of the narrow band light upon absorption and re-emission by the fluorescent molecules is shifted and detectable by the imaging assembly.

12. The system of claim 1, wherein the imaging assembly includes a global shutter configured to control a period of image frame exposure, and wherein the operations further include:
switching between controlling the first light source and the second light source at an end of the period of image frame exposure.

13. The system of claim 1, wherein the imaging assembly includes a rolling shutter configured to control a period of image frame exposure, and wherein receiving the second image frame includes:
receiving a plurality of second image frames, wherein, based on the rolling shutter, a portion of the plurality of second image frames include mixed image frames exposed to the white light and the narrow band light as the computing system switches between controlling the first light source and the second light source; and
identifying and discarding the mixed image frames.

14. The system of claim 1, wherein the first light source is a component of the medical device or the computing system, and wherein the second light source is a component of the medical device or the computing system.

15. The system of claim 1, wherein the system is operable in a white light imaging mode, a fluorescence imaging mode, and a hybrid imaging mode.

16. A computing system communicatively coupled to a medical device, comprising:
at least one memory storing instructions; and
at least one processor coupled to the at least one memory for executing the instructions to perform operations, the operations including:
receiving a first image frame of a target area captured by an imaging assembly of the medical device as a first light source is emitting white light onto the target area;
receiving a second image frame of the target area captured by the imaging assembly as a second light source is emitting narrow band light onto the target area;
dividing the first image frame into a plurality of first tiles and the second image frame into a plurality of second tiles;
for each tile of the plurality of first tiles and the plurality of second tiles, determining a sum of amplitudes for first, second, and third pixels within the respective tile;
comparing shifts in the sum of amplitudes across the plurality of second tiles to identify a subset of the plurality of second tiles corresponding to a region of interest within the target area; and
using shifts in the sum of amplitudes across the plurality of first tiles as a control to confirm the subset of the plurality of second tiles correspond to the region of interest.

17. The computing system of claim 16, the operations further including:
processing the first image frame to generate a white light image;
processing the second image frame to generate a fluorescence image; and
providing the white light image and the fluorescence image to a display device communicatively coupled to the computing system for simultaneous display.

18. The computing system of claim 16, the operations further including:
processing the first image frame to generate a white light image;
processing the second image frame to generate a fluorescence image;
blending the white light image and the fluorescence image to generate a combined image; and
providing the combined image to a display device communicatively coupled to the computing system for display.

19. The computing system of claim 16, the operations further including:
processing the first image frame to generate a white light image;
identifying a boundary of the region of interest based on the comparing of the shifts in the sum of amplitudes across the plurality of second tiles;
smoothing the boundary; and
overlaying the boundary on the white light image to generate an annotated white light image.

20. A method performed by a computing system communicatively coupled to a medical device, the method comprising:
receiving a first image frame captured by an imaging assembly of the medical device as a first light source is emitting white light onto a target area;
receiving a second image frame captured by the imaging assembly as a second light source is emitting ultraviolet light onto the target area to excite any fluorescent molecules present within the target area that are indicative of a region of interest;
dividing the first image frame into a plurality of first tiles and the second image frame into a plurality of second tiles;
determining a first intensity profile across the plurality of first tiles and a second intensity profile across the plurality of second tiles;
comparing shifts in the second intensity profile to identify a subset of the plurality of second tiles corresponding to the region of interest; and
using shifts in the first intensity profile as a control to confirm the subset of the plurality of second tiles correspond to the region of interest.
